(19) United States
(12) Patent Application Publication
(10) Pub. No.: US 2024/0404693 A1
(43) Pub. Date: Dec. 5, 2024

(54) IMPLICIT HEALTH MONITORING VIA SMARTPHONE

(71) Applicant: Carl Zeiss Meditec AG, Jena (DE)

(72) Inventors: Christoph SCHAEFF, Oberkochen (DE); Stefan SAUR, Oberkochen (DE); Wolfgang UNTERGEHRER, München (DE)

(21) Appl. No.: 18/677,247

(22) Filed: May 29, 2024

(30) Foreign Application Priority Data
May 31, 2023 (DE) 102023114284.4

(51) Int. Cl. G16H 40/67; G16H 20/00; H04W 4/38

(52) U.S. Cl. CPC G16H 40/67; G16H 20/00; H04W 4/38

(57) ABSTRACT

A method for monitoring vital data of a user of a mobile device is presented. The method here comprises the following: acquiring environment measurement data in relation to the mobile device by means of a sensor that is fixedly integrated in the mobile device, acquiring interaction measurement data of the user with the mobile device by means of a sensor, wherein the sensor is fixedly integrated in the mobile device, wherein the interaction measurement data are acquired if predefined conditions in relation to the acquired environment measurement data are met, storing the interaction measurement data, determining trend data on the basis of the acquired interaction measurement data for a configurable time period, and generating a signal when a condition is met which is selected from exceedance of a first configurable threshold value regarding the determined trend data, and exceedance of a second configurable threshold value regarding one of the acquired interaction measurement data, in order to monitor the vital data of the user.

### CROSS-REFERENCE TO RELATED APPLICATIONS

The present application claims priority under 35 U.S.C. § 119 to German Patent Application No. 10 2023 114 284.4, filed May 31, 2023, the contents of which are incorporated by reference herein in their entirety.

### FIELD OF THE INVENTION

The invention relates to a computer-implemented method for monitoring vital data of a user of a mobile device. The invention furthermore relates to a monitoring system for monitoring vital data of a user of a mobile device, and to a corresponding computer program product.

### TECHNICAL BACKGROUND

Monitoring vital parameters of patients in a medical center or hospital has already been part of everyday clinical practice for a long time. Moreover, long-term measuring devices for specific vital parameters—for example long-term blood-pressure measuring device, blood sugar measuring device—have been known for a relatively long time. However, measurement outside a clinical environment typically proves to be complex and cost-intensive.

In contrast to fractures or infarcts, many physical and/or mental diseases become established as a result of an insidious, continuous process that usually lasts for years. In order to detect these syndromes (e.g. presbyopia or loss of hearing) at an early stage, periodic monitoring of the corresponding body functions is necessary. However, hitherto this has always necessitated explicit testing by way of a measuring method—whether on the part of a physician/optician or via self-tests (e.g. sight test on a smartphone) or at home. This explicit testing has the disadvantage, however, that it needs to be initiated directly by the user and is therefore often forgotten or neglected (“one feels well after all”). Therefore, the syndromes are often not diagnosed until at an overly late stage. Moreover, a review of the healthy state or the development history (e.g. speed of progression) is no longer possible.

There is therefore a need to be able to determine the physical and/or mental fitness implicitly and continuously over a relatively long time period. Indicators of an insidious disease progression can thereby be ascertained at an early stage. A preventive treatment can also be diagnosed in this way.

### OVERVIEW OF THE INVENTION

This object is achieved by means of the method proposed here, the corresponding monitoring system and the associated computer program product as claimed in the independent claims. Further configurations are described by the respectively dependent claims.

According to a first aspect of the present invention, a computer-implemented method for monitoring vital data of a user of a mobile device is presented. The method here comprises acquiring environment measurement data in relation to the mobile device by means of a sensor, wherein the sensor is fixedly integrated in the mobile device, acquiring interaction measurement data of the user with the mobile device by means of a (at least one) sensor that is fixedly integrated in the mobile device, wherein the interaction measurement data are acquired if predefined conditions in relation to the acquired environment measurement data are met, and storing the interaction measurement data.

Furthermore, the method comprises determining trend data on the basis of the acquired interaction measurement data for a configurable time period, and generating a signal when a condition is met which is selected from exceedance of a first configurable threshold value regarding the determined trend data, and exceedance of a second configurable threshold value regarding one of the acquired interaction measurement data, in order to monitor the vital data of the user.

According to a further aspect of the present invention, a monitoring system for monitoring vital data of a user of a mobile device is presented. The system comprises a processor and a memory which operatively cooperates with the processor to store instructions which, when executed by the processor, cause the processor to implement acquiring environment measurement data in relation to the mobile device by means of a (at least one) sensor that is fixedly integrated in the mobile device, and acquiring interaction measurement data of the user with the mobile device by means of a sensor that is fixedly integrated in the mobile device. In this case, the interaction measurement data can be acquired if predefined conditions in relation to the acquired environment measurement data are met.

Furthermore, the processor is adapted for storing the interaction measurement data and for determining trend data on the basis of the acquired interaction measurement data for a configurable time period, and for generating a signal when a condition is met which is selected from exceedance of a first configurable threshold value regarding the determined trend data, and exceedance of a second configurable threshold value regarding one of the acquired interaction measurement data, in order to monitor the vital data of the user.

The proposed computer-implemented method for monitoring vital data of a user of a mobile device has a plurality of advantages and technical effects which may also apply accordingly to the associated monitoring system:

The use of a mobile device, in particular a smart device such as a smartphone, for example, which a user typically carries around with them every day, and the sensors installed therein, enables an implicit determination of changes in the physical and/or mental abilities practically at no cost and effortlessly by way of the observation of the use of the smartphone. In this case, the advantages are not restricted to the use of a smartphone, but rather can also be realized by way of an observation (i.e. measurement) of the interactions with a PC, a laptop, a tablet computer, a phablet, a smartwatch, etc.

In other words, the patient or the physician is not restricted to explicit measuring methods that have to be triggered individually. Rather, an app of the mobile device—or as a function that is integrated directly in an operating system of the mobile device—can continuously or periodically acquire sensor data and/or input data, evaluate them and compare them with historical data in order to derive a trend regarding a development of the physical and/or mental fitness over a time period. A signal can be generated when limit values are exceeded or undershot, the limit values being definable in absolute or relative terms. The relativity can be achieved e.g. by reference to a demographic group. The signal can be advice to the user or dedicated advice to visit a (specific) physician or optician for a specific treatment. A potential side effect is dedicated, but nevertheless indirect, advertising for the physician or optician.

Advantageously, here by way of example the following measurements and potential pieces of advice and/or diagnoses can be provided continuously:
an analysis of the volume of the loudspeaker/audio output can afford an indication of hardness of hearing if the trend reveals an increase in the volume.
an analysis of the distance “between eye and mobile device” (e.g. using camera/depth sensor) can afford an indication of short-sightedness if the trend reveals an increase in the distance.
an analysis of the brightness of the display of the mobile device in relation to the ambient light or a brightness tolerance, or vision against the light, can be an indication of an eye disease.
an analysis of the frequency of the blinking/screwing up of an eye (e.g. acquired using a camera of the mobile device) can represent an indication of dry eyes.
an analysis of the reflex response or the speed of reaction to optical stimuli (e.g. during a video game) or new information, also depending on location, can be an indication of visual spots or some other neurophysiological degeneration.
an analysis of a movement pattern of the mobile device (e.g. using an IMU sensor) can suggest indications of orthopedic diseases if limping is suddenly discernible during walking or the sitting posture changes.
an analysis of the vocabulary and/or typing speed (e.g. during an input via the (display) keyboard) can be an indication of schizophrenia or Alzheimer's disease (e.g. relatively long pauses during the typing of personal names).
an analysis of general user behavior (e.g. number of actively used or new apps per year) can represent an indicator of the mental fitness of the user.
an analysis of gyroscope data during the typing of messages can enable an indication of tremor.
an analysis of screen use (e.g. “are there areas that are never looked at or where the mobile phone is then physically moved in order to read them?” by means of eye tracking with the aid of a camera and IMU sensor data) can afford an indication in relation to visual spots.
an analysis of zoom behavior concerning the displayed font size (e.g. font size 10 is read only with zoom of 110% after 5 years, and previously at 90%) can be an indication of a deteriorating visual faculty.
an analysis of a general movement profile (ascertained by means of an IMU sensor) with simultaneous use/fixation on the screen (e.g. by means of eye tracking with the aid of a camera) can be an indication of impaired balance.

In the case of all such measurements and indications of potential diseases, it is possible to have recourse exclusively to the sensors in the mobile device.

Moreover, the presented concept makes it possible to avoid measurements or analyses that lead to incorrect assessments. In the case of continuous data acquisition over a relatively long time period in uncontrolled environments (e.g. with the user not sitting rigidly and reproducibly in front of an ophthalmological or acoustic measuring device), it should be taken into consideration, however, that potential sources which can generate a systematic bias in the measurement data must be compensated for or taken into account in some other way. This may happen if the measurement data were not recorded in the expected environment or with the expected frequency distribution.

There may be various sources of this systematic bias: (i) the user uses their smart device primarily while standing rather than—as expected—while sitting for an analysis of visual behavior; (ii) the user uses their smart device primarily in commuter traffic—for example when crowded together in the underground—i.e. the user can only use the device sub-optimally (e.g. “head/screen” distance); (iii) the user uses their smart device primarily in an environment with loud background noise that would need to be taken into account in acoustic analyses; (iv) the user uses their smart device primarily outdoors in daylight, which would need to be compensated for in visual analyses.

Moreover, in the case of a model change for the smart device, if appropriate, a normalization or scaling of the data by specific correction factors could also be carried out if e.g. the display resolution of a camera changes or the measurement scales of the individual sensors change. Moreover, in the case of a continuous long-term measurement, relatively large amounts of data are generated, which would additionally need to be stored, transmitted and/or processed further.

The concept presented here addresses all these negative influences in an elegant way and thus enables a periodic acquisition of measurement data, which however can be effected implicitly—i.e. without a direct activation by the user. In this case, a controlled environment of the measurement recordings is ensured in order to avoid bias and the trend analyses make it possible to significantly reduce the amounts of data that are to be processed or transmitted by comparison with traditional continuous long-term measurements (i.e. measurements after respective discrete intervals).

Developments of the invention are presented below, which can have validity both in association with the method and in association with the corresponding monitoring system.

In accordance with one advantageous embodiment, the method can furthermore comprise reducing the interaction measurement data on the basis of a rule. The reducing can also comprise a selection regarding the amount, e.g. the number of measurement values. This can be based on a function—for example a selection function. The reducing can preferably be effected in or for a configurable time interval, wherein the rule(s) can also be configurable. Furthermore, the environment measurement data can also be reduced in terms of their amount. Both for the interaction measurement data and for the environment measurement data, the reduction can be effected by an approximation function and/or a trend function being formed from the respective measurement data, such that only the function parameter values have to be stored. Furthermore, measurement value outliers both in the environment measurement data and in the interaction measurement data can also be removed according to predefined rules. Overall, the reducing has a positive effect on the volume of data that has to be stored.

In accordance with one interesting embodiment of the method, the acquired interaction measurement data can be indicative of physical and/or mental vital parameter values of the user—in particular of the bearer of the mobile device or the patient. This can result in elegant continuous monitoring of the state of health on the basis of the vital parameters. Alarms, in particular for medical emergency services, can also be generated in this way. These can be realized by automatically generated signals of the mobile device to an emergency response control center.

In accordance with further embodiments of the method, the interaction measurement data can comprise at least one from the group consisting of the following:
an amplitude (or more generally “signal strength”) of an audio signal of the mobile device (for example of a microphone, loudspeaker, headphones),
a frequency distribution of an audio signal of the mobile device during an active use of the mobile device,
retrieval of optically perceptible data,
active operating, such as scrolling through a document or a web page,
a brightness of a screen signal of the mobile device (e.g. in comparison with the ambient light of the mobile device),
a frequency of eye blinking by the user (e.g. detected using a camera of the mobile device),
a speed of the user's reaction to optical stimuli which originate from the mobile device and which are preferably emitted in a targeted manner,
an attention duration (e.g. detected by way of a scrolling speed, drifting of the pupil away from the screen, etc.),
a speed for inputting a text or an individual word or individual letters,
a number of (simultaneously) actively used apps,
a rectilinearity of acquired swiping movements,
a frequency of use of the mobile device,
screen areas of the mobile device that are used by the user, and
a zoom setting of the mobile device.

Other measurement data of the user of the mobile device can additionally be acquired. However, e.g. movement profiles, whereabouts, etc. should preferably be acquired as environment measurement data since they do not relate directly to an interaction between the user and the mobile device, but rather are acquired by potentially other measuring sensors of the mobile device that are not geared to the direct interaction between the user and the device.

Moreover, it should be mentioned that the stated distance between the mobile device and the head can in particular also relate to the distance between the mobile device and the user's face. Ascertaining the distance between the mobile device and the user's head can comprise a distance measurement between the mobile device and the user's face. Using software for recognizing anatomical features, it is possible to automatically recognize for example eye(s) and/or ear(s) of the user in the camera image of the mobile device. Using depth or autofocus sensors of the mobile device, it is thereby possible for distances between selected anatomical features of the head and the camera of the mobile device to be acquired precisely.

In accordance with an additional embodiment of the method, the environment measurement data can comprise at least one from the group consisting of the following: ambient brightness values, ambient volume values, kind of represented information of the mobile device (in particular on a screen of the mobile device, characteristics regarding a represented text or a viewed video or an interactive game), a spatial orientation of the mobile device (i.e. how the device is held), a speed of movement of the mobile device, a movement profile of the mobile device, GPS coordinate values (or from other locating services), and a camera image.

Acquiring the speed of movement of the mobile device can make it possible for example to draw a distinction regarding stationary behavior (e.g. sitting or lying), an automobile journey (on the basis of the speed), a flight, a train journey, etc., with respect to the user. The requisite acquisition can be effected using a gyroscope integrated in the mobile device and/or acceleration sensors integrated in the mobile device. In this case, it is also possible to draw a distinction between large-scale movements or small-scale movements of the mobile device. Large-scale movements relate rather to the movement of the mobile device and thus the user overall, while small-scale movements may be indicative rather of the movement of the hand holding the mobile device. Such an acquisition can be information with regard to an orthopedic disease such as Parkinson's disease, etc. Various measurement values can be acquired for this purpose, such as e.g. frequency of movement, rapidity of movement, temporal length of the respective movement (e.g. sporadic shaking). Moreover, the fact of whether the user is located in a building, outside in the open, etc. can be acquired in this context.

The whereabouts can be located for example using GPS coordinate values (or using other locating services). An evaluation of WLAN access point data is also possible inside buildings. With regard to the camera image, either a front-side camera of the mobile device or a rear-side camera (or cameras) can be used. Preferably, e.g. by way of image recognition, it is possible to establish whether or not the user is wearing spectacles or contact lenses at the time when interaction measurement data are obtained.

In accordance with one supplementary embodiment, the method can additionally comprise allocating a predefined profile of interaction measurement data—and associated environment measurement data in a further supplementary embodiment—to at least one characteristic of vital parameter values. In particular, areas of interaction measurement data of a predefined profile can be allocated to the characteristic of vital parameter values. This characteristic of the vital parameter values can relate in particular to syndromes and/or at least one fitness characteristic.

For this purpose, the environment measurement data need not necessarily be included—but may optionally be included. Trend data of the interaction measurement data could be sufficient for a statement about the vitality of the user.

For the case where the interaction measurement data (optionally in combination with the environment measurement data) include indications about a particular syndrome, e.g. a (warning) signal can also be output. Furthermore, it is also possible to effect a display on the screen of the mobile device, comprising for example a recommendation in the form of an action instruction. The action instructions here in the sense of action recommendations can encompass a very broad spectrum, e.g. go to the physician in at least two months. The action recommendation to refrain from driving a motor vehicle can be given if examination of eyesight reveals that it falls below a specific level (e.g. 50%) with high probability, and/or that a visit to an optician is recommendable. These action recommendations or action instructions can be selected automatically from a set of predefined action instructions depending on the interaction measurement data—optionally also depending on the environment measurement data.

Furthermore, interaction measurement data can relatively quickly signal an increase in the user's short-sightedness. Furthermore, environment measurement data can draw attention to extensive screen work over a relatively long time period. Additionally, it is also possible to detect absence of sufficient recovery phases and additionally sleep deprivation. A subsequent recovery of the visual faculty (demonstrated by the interaction measurement data) during a relatively long holiday (demonstrated by the environment measurement data) may lead to the conclusion that the vital parameter values are okay and there are no signs of a pathological change in the visual faculty. There may be a similar situation for temporary eye blinking.

In accordance with one further developed embodiment, the method can comprise actively instructing the user, by means of the mobile device, to fulfil a predefined use profile in relation to the mobile device if predefined interaction measurement data have not been acquired within a predefined time interval. Such active instructions for the user may be, for example, adopt a sitting position, read a sample text with a predefined font and zoom factor, create specific ambient lighting conditions, set a predefined display brightness, use a specific position for holding the mobile device, etc. Moreover, the respective requested use profiles may demand a predefined measurement duration during which the user should comply with the corresponding requested use profile.

In accordance with another further developed embodiment of the method, actively instructing can take place if the environment measurement data for the acquisition of the interaction measurement data are in a predefined value range. This makes it possible to ensure that the time at which instructing takes place is preferably if current environment measurement data are close to a predefined value spectrum, or if individual data categories of the environment measurement data—in particular those that are the most important in accordance with a predefined order—are fulfilled in a prioritization. The following can be specified as examples: “sitting in a specific time range” (e.g. after getting up, in the morning, in the afternoon, in the evening, etc.), not when moving around and/or driving an automobile, but rather if the environment measurement data are almost optimally fulfilled. This could significantly increase acceptance by the user. Moreover, it is possible in this way to relax a rather strict catalogue of criteria (i.e. the predefined conditions) for environment measurement data which are a deciding factor in acquiring the interaction measurement data if it is established that the user is at rest, that is to say e.g. “on the sofa/couch”.

In accordance with another elegant embodiment, the method can provide for reducing a number of interaction measurement data—in particular of the same type—assigned to one another if a change in interaction measurement data assigned to one another (e.g. within a time series) is below a predefined threshold value. This can lead to a thinning out of the trend support data—i.e. to a reduction of measurement data—if no or only small changes in successive measurement data of a series are established. In this case, it is also possible to carry out an active change—for example based on a rule—in a set of interaction measurement data associated with one another if a predefined number of interaction measurement data are below a respectively predefined threshold value.

This can preferably be done if a representative value (e.g. an average value or a value of a weighted average) has been determined for a stored amount of interaction measurement data that have been ascertained e.g. within a period of time, or simply after a configurable time period has elapsed.

In accordance with one advantageous embodiment, the method can furthermore comprise acquiring an input of a situation indication into the mobile device, wherein the situation indication is selected from a selection of situation indications provided by the mobile device. The user can effect the input, with the situation indication concerning the user themself. In this case, a linkage of the situation indication that is selectable and selected by the user with a set of interaction measurement data acquired at a specific point in time can be produced. In addition, free inputs concerning the user's situation are also possible.

In accordance with one supplementary embodiment, the method can also comprise quasi-continuously measuring—preferably also at small intervals—a distance between the screen of the mobile device and an eye of the user within a predefined time period and/or while previously determined environment measurement data values are present, and using different tilt angles of the mobile device for determining a reference distance between at least one eye of the user and the screen surface. The different tilt angles can be acquired here by the quasi-continuous measuring e.g. by means of the device-internal gyroscope. The reference distance here is a distance value which can be formed from a plurality of measurements carried out at different tilt angles. For example, the reference distance can be a mean value of the measurements, wherein the individual measurements can be weighted equally or differently. The reference distance can e.g. be ascertained separately for each eye and be applicable to one or both eyes.

The previously determined environment measurement data values can comprise a lower limit and/or an upper limit of a value for a specific category of environment measurement data. By way of example, the distance is measured only if a minimum value of an ambient brightness is complied with or a movement of the mobile device takes place with an acceleration value below a maximum value.

In accordance with one extended embodiment, the method can additionally comprise determining a quality value of the acquired interaction measurement data, which e.g. is indicative of a quality of the data, and repeating the acquisition of the interaction measurement data if the quality value is below a predefined quality reference value. The quality value can give indications about various parameters of the image recordings, for example. Examples are: the quality of the focusing of the camera, e.g. the degree of defocusing of a point or object to be focused on, blur owing to an excessively long exposure time relative to a movement of the mobile device and/or of the object for recording, image noise owing to an ISO level being chosen to be too high, on account of excessively low ambient brightness, for example, contamination of the camera optical system, or, unlike what is envisaged or customary, the user is not wearing a visual aid that would be necessary for uniformly registering vision. The quality reference value is e.g. a value indicating the threshold as of which a quality of the interaction measurement data is sufficient or not sufficient for a further use. This can be ascertained e.g. in tests to be carried out beforehand.

In accordance with another further developed embodiment, the method can moreover comprise using an acquisition profile for acquiring the environment measurement data and/or acquiring the interaction measurement data. For example, it can be informative to know at what times relative to specific user activities measurement is performed. The acquisition profile, for acquiring the interaction measurement data and/or environment measurement data, can be determined at one or a plurality of regularly recurring points in time which can have a fixed relation to a user behavior. The acquisition profile used can be a recurring appointment or an appointment type which can be manually defined by the user or can be proposed by a program on the basis of a user profile ascertained beforehand and can be selected by the user. The recurring appointment or the appointment type can take place e.g. once or a number of times weekly at a specific time of day. By way of example, that can encompass characteristic times with regard to strain or relaxation of a user, such as e.g. Sunday evening as a characteristic point in time for relaxation (in the case of a 5-day working week starting from Monday) or Friday morning as a characteristic point in time for strain (at the end of such a working week). This can depend on a user-specific daily/weekly routine. For example, a physician who works shifts may have a different user profile than an administrative employee for an authority or someone who works part-time or has retired. Depending on the kind of configured recurring appointment or appointment type, by way of example, it is possible to determine a mean value from extrema in relation to strain and/or it is possible to deduce the user's individual regenerative capacity by way of measurements at times of strain and relaxation.

In accordance with one supplementary embodiment of the method, standardizing the interaction measurement data—through the use of the acquired environment measurement data—can make them at least partly independent of environmental influence data. In other words, it is possible to reduce a bias in a set of interaction measurement data recorded over a relatively long time period.

Moreover, in accordance with one additional exemplary embodiment of the method, provision can be made for automatically erasing—if appropriate by overwriting in the memory—interaction measurement data and/or environment measurement data in accordance with a previously defined rule. In this way, (restricted) memory space available in the mobile device can be used in an optimized manner.

### OVERVIEW OF THE FIGURES

Preferred exemplary embodiments of the present invention are described by way of example and with reference to the following figures:

FIG. 1 shows a flowchart-like representation of one exemplary embodiment of the method according to the invention.

FIG. 2 shows a sequence, more akin to implementation, of one exemplary embodiment of the proposed concept.

FIG. 3 shows a diagram of one exemplary embodiment of the monitoring system.

FIG. 4 illustrates one exemplary embodiment of a computer system that comprises the monitoring system according to FIG. 3.

### DETAILED DESCRIPTION OF THE FIGURES

In the context of this description, conventions, terms and/or expressions should be understood as follows:

The term “mobile device” or smart device can be any electronic device which a user usually carries with them. This is typically a smartphone. Alternatively, however, it can also be a dedicated device provided exclusively for the concept presented here. Alternatives thereto can also be a smartwatch, a phablet (mixture of smartphone and tablet computer), a tablet computer, a notebook computer or so-called goggles, which are worn in a similar manner to spectacles but in which additional information is projected onto the “spectacle lenses”. Moreover, other dedicated portable electronic devices are also possible as mobile devices within the meaning of the present text. It may be the case that not all of the functions described here are realizable by all of the devices mentioned by way of example. However, all of the mobile devices mentioned here typically have a plurality of sensors and/or actuators, such as, for example, microphone, loudspeaker, acceleration sensor, camera(s), pose sensor, and in some instances also temperature sensors, air pressure sensors and/or position sensors (for example on the basis of GPS [Global Positioning System] or Glonass or Galileo).

Finally, it should be pointed out that the functions of the concept presented here can also be realized as a software application—e.g. as an app on the mobile device—or else—in the case of a dedicated device—as a pure hardware solution or a combination of hardware and software.

The term “sensor”—in particular sensor of the mobile device—describes a measuring unit which can record and optionally also store physical measurement values. These measurement values are typically digitized in order to make them more easily processable by means of software.

The term “user” here describes that person who predominantly carries the mobile device with them and uses it.

The term “environment measurement data” describes measurement data which are measured by one or more sensors of the mobile device and do not directly relate to the user. The environment measurement data can thus be e.g. data about an audiovisually acquirable environment of the mobile device and/or whereabouts and/or movement.

The term “interaction measurement data” here describes measurement data which are also measured by one or more sensors of the mobile device, but which relate directly to the user or an interaction between the user and the mobile device.

The term “trend data” here can characterize or describe a progression of a series of measurement values of the interaction measurement data over a definable period of time. This can be implemented e.g. by means of a function with reference to the interaction measurement data. In this case, the function can use the measurement values of the interaction measurement data to a definable approximation and/or fineness of a gradation over time. In one exemplary embodiment, a regression straight line (i.e. linear regression) can be used for this purpose. In other exemplary embodiments, higher-order polynomials are also possible. The trend at a given point in time can then be defined e.g. by the first derivative of the respective function at the given point in time.

The term “storing” of any measurement data here describes at least temporary storing. Storing can take place in various ways and at various locations, e.g. locally on the mobile device and/or in a cloud storage facility. Moreover, all calculations or just portions thereof can also be effected by a cloud computing system. In this case, the mobile device would be used for acquiring the measurement data and for transmitting the measurement data to the corresponding cloud computing service(s).

The term “vital data” describes data reflecting basic functions of the human body. In the medical field, vital data are also often referred to as vital parameters. They often include heart rate, blood pressure, body temperature and respiratory rate. In the context of the present text, predominantly other data belong to the user's vital data. They can also be derived data which are not directly measured. One example would be the speed at which texts are input on a keyboard of a smartphone. For example, conclusions about the user's physical and mental fitness can be drawn therefrom. Another example can be the reading speed and hence the scrolling speed for a text on a screen of the smartphone. By way of the chosen font size of texts presented on the screen of the smartphone, e.g. conclusions about the user's visual faculty can be drawn.

The term “signal” can be any indication that is perceptible by a user. Signal combinations can also be used. For example, a text output can be accompanied by an acoustic signal or a vibration alert. There can also be a voice output, which may first need to be activated by the user. The trend data with potential instances of threshold values being exceeded or undershot can be displayed graphically. In principle, all signal systems of the mobile device can be used.

A detailed description of the figures is given below. It is understood in this case that all of the details and instructions in the figures are illustrated schematically. A flowchart-like representation of one exemplary embodiment of the method according to the invention for monitoring vital data of a user of a mobile device is presented initially. Further exemplary embodiments, or exemplary embodiments for the corresponding monitoring system, are described subsequently:

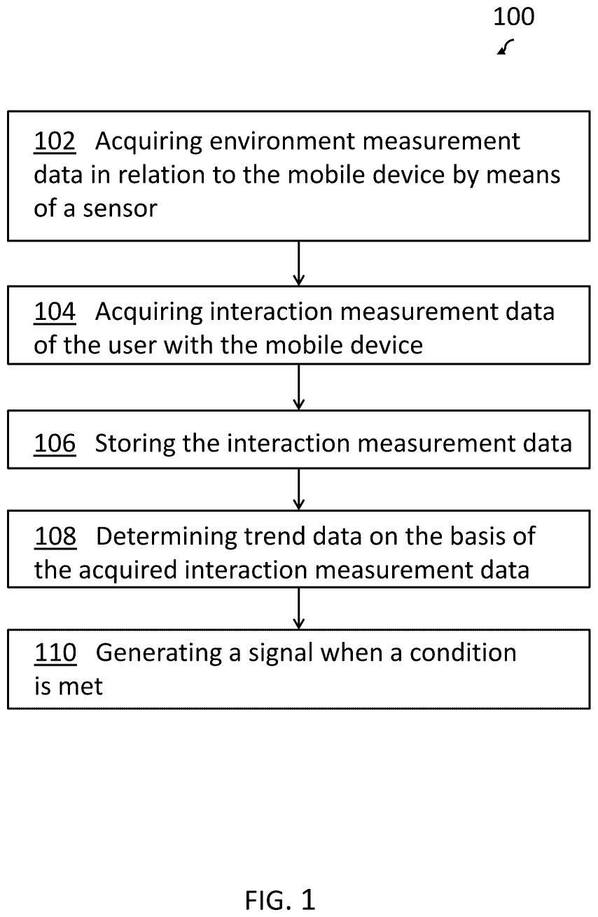

FIG. 1 presents a flowchart-like representation of one preferred exemplary embodiment of the computer-implemented method 100 for monitoring vital data of a user of a mobile device, i.e. for example a smart device, smartphone, smartwatch, tablet computer.

In this case, the method 100 comprises acquiring, 102, environment measurement data—i.e. in particular data of the immediate environment measured by way of sensors of the mobile device, optionally together with a date/time stamp—in relation to the mobile device by means of at least one sensor. The sensors for acquiring the measurement values are typically installed fixedly with or in the mobile device. Alternatively, however, it is nevertheless possible for the mobile device to be extended by particular further measuring units—i.e. sensors. These can be connected to a USB interface of the mobile device via an adapter, for example.

The method 100 furthermore comprises acquiring, 104, interaction measurement data. In this case, the interaction measurement data are those which are ascertainable as a result of the interaction between the user and the mobile device by means of at least one sensor. The sensor(s) need not be the same as that/those used for the measurement of the environment measurement data. A dual use of sensors is nevertheless possible. Here, too, the sensors should be fixedly connected to the mobile device. They should thus primarily be standard sensors of the smartphone, for example. The smartphone can nevertheless be extended by further sensors for additional measurement value parameters.

The sensor(s) for the interaction measurement data and the environment measurement data can differ from one another; however, they can also be identical. If there are a plurality of sensors, subgroups of the respective sensors can also overlap functionally. One example would be a use of the front camera for example to ascertain a distance to the user's eye (i.e. interaction measurement data) and a simultaneous (or in the temporal context) determination of the ambient brightness (i.e. environment measurement data).

Preferably, the recording of interaction measurement data is effected quasi-continuously or periodically and need not normally be explicitly initiated by the user (although this would be additionally possible). The actual acquisition—in particular storage of the interaction measurement data—is usually effected only if predefined conditions in relation to the acquired environment measurement data are met. By this means, firstly, it is possible to avoid specific measurement data corruption (i.e. bias)—e.g. as a result of non-representative measurement times. Secondly, it is also possible for example to avoid the recording of interaction measurement data within relatively short intervals, e.g. during the user's sleep. This would just require energy and memory space unnecessarily.

The method 100 moreover comprises storing, 106, the interaction measurement data. Additionally, environment measurement data measured at the same time can be acquired and also stored. The measurement interval of the environment measurement data can also be chosen to be larger or smaller in comparison with the measurement interval of the interaction measurement data. In this way, the two classes of measurement data can be placed in a shared context. In this case, the data do not have to be stored permanently. Compression of the data is possible in order thus to save memory space in the mobile device. Moreover, wireless transmission of the data to stationary memories and evaluation units is possible.

Furthermore, the method 100 comprises determining 108 trend data on the basis of the acquired interaction measurement data—and optionally also on the basis of the acquired environment measurement data—for a configurable time period. The trend data can be derived from a plurality of acquisitions of interaction measurement data in a predefined time interval, wherein the acquisitions are preferably provided for appointment types within the time interval, such that it is possible to determine further support points for generation for the trend data for a respective appointment type.

Additionally, the method describes generating 110 a signal if a condition is met. The condition can be exceedance of a first configurable threshold value regarding the determined trend data, or exceedance of a second configurable threshold value regarding one of the acquired interaction measurement data, in order to monitor the vital data of the user. In this case, trend data can also be taken into account accordingly. The term “exceedance” should be understood with a double meaning here, namely with regard to exceedance of a lower limit or of an upper limit. In this respect, it is also possible to state that a signal can be generated if a set limit value is undershot.

The signal can have various forms, for example optical, acoustic, haptic, or a combination thereof. This can involve a direct voice output of the mobile device, or the user is made aware of the voice output by means of a connected device—e.g. Bluetooth loudspeakers. The condition can also simply reside in the user of the mobile device retrieving corresponding data from the memory of the mobile device.

Other forms of a signal would be a text output and/or an optical indication (marker) in a represented graphic of the measurement values, for example on the display of the smartphone.

Moreover, for generating the signal a second condition should be necessary, for example exceedance—or undershooting—of a second configurable threshold value regarding one of the acquired interaction measurement data, in order to monitor the vital data of the user.

That is to say, in other words, that a signal is generated if the (determined) trend data exceed a first configurable threshold value or if the acquired interaction measurement data exceed a second configurable threshold value.

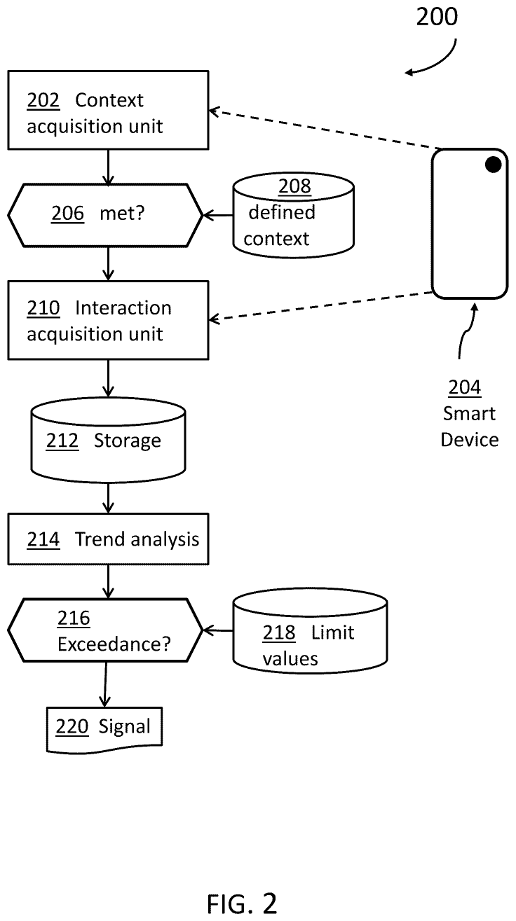

FIG. 2 shows a sequence 200, more akin to implementation, of one exemplary embodiment of the proposed concept. After an activation (not illustrated), a context acquisition unit 202 can be activated periodically or at configurable time intervals after a last measurement data acquisition, in order to acquire environment measurement values by means of one or more sensors of the smart device (e.g. smartphone) 204 as mobile device. After an evaluation (either directly in or near the sensor or directly in the smart device), the environment measurement values are then processed to form environment measurement data.

This is followed by checking, 206, whether the acquired environment measurement data are in a predefined area—i.e. a predefined context 208. These pieces of context information are retrieved from a memory of the smart device. Alternatively, these can also be retrieved or regularly updated by a remotely installed device via a wireless interface. If the condition of the predefined context 208 is met, interaction measurement data can be acquired by an interaction acquisition unit 210, the latter at least also including a sensor, and can optionally be stored, 212. This at least one sensor need not be the same as for the acquisition of the environment measurement data. Data values are typically acquired by a plurality of sensors. A joint use of the sensors of the context acquisition unit 202 and of the interaction acquisition unit 210 can also occur here.

There then follows a data analysis—e.g. trend analysis 214—regarding the measured interaction measurement data. If exceedance 216 with predefined limit values 218 is established, the signal 220 is generated, which draws the user's attention to carrying out a specific action (e.g. in order to activate a new measurement or to plan a visit to a physician).

A wide variety of scenarios can be addressed with these method steps or this arrangement of a corresponding monitoring system. This shall be explained on the basis of the example of a context acquisition with accompanying bias compensation for a long-term analysis of the “eye/smartphone” distance.

During the long-term analysis of the distance between the user's eye and their smartphone or their smart device, it is necessary, in order to recognize a development of short-sightedness at an early stage, to take account of various potential sources which can produce a bias. The bias can advantageously be compensated for by a simultaneous acquisition of the context in the corresponding measurements or a predefinition of requirements in respect of the context:

The acquisition of the user (e.g. by way of the image from the front camera of the smartphone) makes it possible to exclude sensor measurement data (either directly during recordings or during post-processing) which possibly originate from other users of the smartphone.

An acquisition of IMU data (IMU=Inertial Measurement Unit, which can consist of a plurality of acceleration sensors, for example) as environment measurement date can make it possible to include in the analysis only such sensor measurement data in which the user was not in motion, for example. This can take account of the fact that the smartphone is generally held differently during walking compared with when the user is in a sitting position, since the environment needs to be observed as well.

Furthermore, an acquisition of an image by way of the main camera and/or a use of GPS data makes it possible to determine the spatial context (e.g. in the place of residence, in the automobile, . . . ) in which the sensor data are acquired. Depending on whether the long-term analysis then requires statistical measurement data regarding a broad or narrow spatial context, the sensor measurement data can be recorded accordingly. In other words, the acquisition of the interaction measurement data can be controlled in a context-dependent manner.

This example already reveals in an exemplary fashion that, for each potential syndrome, firstly the preferred context as a predefined use profile—in the above example, therefore, resting, sitting position, in daylight, in the interior, when reading relatively long texts, without disturbing light reflections on the display, the user is using already available spectacles/contact lenses in order that the method according to the invention can assess the function of the visual aid—and a minimum measurement interval (for example one month) can be defined and stored in the smartphone. The app in the smartphone then checks at discrete-continuous intervals whether the context conditions are each met. This is done on the basis of sensor measurement data and/or app use data.

This can be concretized even further on the basis of a further exemplary embodiment. In the present case, i.e. if the context is fulfilled, the IMU sensor would detect no acceleration and the orientation of the smartphone would be at an angle of zero degrees (i.e. perpendicular to the Earth's axis) to 45°, which would correspond to a resting, sitting position of the user. Furthermore, the light sensor of the smartphone would measure typical values for daylight, which can additionally take place by way of a coupling with the evaluation of the GPS signal in order to establish whether the user is located in a building. In addition, the screen of the smartphone may display a predefined text—e.g. chat message, email, web page.

If all the context conditions are met, an acquisition of interaction measurement data can potentially be carried out. In order to minimize the amount of data, however, firstly the current date (including time of day) is compared with the date of the last valid measurement. If the difference is less than the stored minimum measurement interval, no measurement is carried out and stored. However, if the difference is greater/greater than or equal to the minimum measurement interval, a measurement is carried out and the associated data are stored and the date of the last valid measurement is updated. This would presuppose that a valid measurement is involved.

Conversely, the corresponding tracking could first be updated if the predefined time interval since the last measurement had elapsed.

In order to check the validity of a measurement, the corresponding measurement evaluation algorithm returns an indication of whether or not the measurement data were able to be successfully evaluated. A negative indication can be present for example in the case of a defocused image, or if it is established by way of image evaluation or after detection of a statistical anomaly with a certain probability that, unlike what is customary, a user has not used a visual aid, to mention just a few examples.

Optionally, provision can also be made for a user, as necessary, to manually add further information (tags) to a performed measurement to supplement the context information acquired anyway. For this purpose, it is also possible to use a voice recognition function together with NLP (Natural Language Processing, i.e. voice recognition) of the smartphone, such that the additional information does not have to be input manually. This can be embodied as selection to be carried out by the user or as confirmation of situation proposals as situation indication which are generated by the app, or as supplementation from a selection if the respective contexts (i.e. data acquisition situations) cannot, or highly probably cannot, be derived from the measurement data. The following are conceivable as examples of generated situation proposals or situation indications: holiday, work, illness, fatigue and/or stress.

As a further option, the app together with the smartphone can also represent the following scenario: depending on the user settings in the app, waiting for the occurrence of a measurement situation defined in advance can be differentiated by a spectrum of measurement situations—e.g. extrema—being acquired in a targeted manner. By way of example, it is possible to ascertain the performance of an eye in the morning/in the evening, on weekdays/at the weekend, with large/small differences in brightness in relation to the display brightness or the ambient brightness. In this regard, it is possible to obtain statements about the adaptability of the eye and a temporal progression can be determined—including separately for the individual measurement times. This can be determined for example by way of data time series for all the measurements on workdays/in the evening (or at other times). As a result, for instance, a requirement spectrum for visual aids (e.g. reading spectacles, spectacles for automobile driving, spread of varifocal spectacles/a multifocal lens) can be determined.

Since the context criteria are not always binary, a probability with which the prepared context is fulfilled can also be calculated. A measurement is carried out if the probability is above a certain threshold value.

If the context criteria have not been satisfied over a relatively long defined time period, if the minimum measurement interval has been exceeded x times, for example, the app can explicitly request the user to meet the conditions of the predefined context. This can take place in a situation for which the context has already been fulfilled to a certain degree. This can be done by way of a notification to actively call up the app, whereupon the user can be requested—in particular for the example of short-sightedness—to read a sample text in a sitting position. A user instruction for producing an optimum measurement situation can also comprise further criteria. Specific ambient/display lighting, predefined holding of the smartphone, a measurement duration, etc. are conceivable for this. The notification produced then appears to be advantageous in particular in combination with the abovementioned optional aspect of acquiring a defined spectrum of measurement situations.

Moreover, a qualification of a change in the vital data (e.g. vision) over a time period of the measurements at intervals and data output on a display of a patient's app are additionally possible. This should then be considered as an analog to long-term step number tracking. The measurements at intervals can be performed optionally in a manner differentiated according to the recording times, which are intended to represent extrema of the performance of the eye.

Furthermore, it is possible to provide for outputting data or notifications to the user in order to output configurable action recommendations if the measurement values are within a defined measurement tolerance. By way of example, the trend assessment may read “no change” if a change in the required diopter number for spectacles is <0.5 diopter, i.e. in line with a threshold value. This corresponds to a decoupling of raw vis-à-vis processed measurement data. An action recommendation “visit optician” or “visit ophthalmologist” can be generated e.g. only starting from a deviation of 0.75 diopter. This then involves a decoupling of processed measurement data and corresponding action recommendations.

In order to satisfy an implementability of the proposed context according to a desired measurement precision, the capability of ascertaining the angle formed between that plane of the smartphone in which the exit pupil of the selfie camera of the smartphone used and the display lie and the user's visual axis appears to be important. That is based on the fact that the autofocus of the selfie camera measures the distance between sensor and eye. By contrast, the distance between display and eye or a reference geometry is probably not measured, but rather defined. In the case of a large smart device (e.g. phablet), a tilt of the device in the region of the lens may constitute a few millimeters, which adversely affects the precision of the distance measurement and thus the assessment of the visual faculty if the assumed reference geometry is not correct. This would actually be the case for a large smart device, where a large distance between the lens, normally arranged at the top of the device, and the displayed text typically occurs. If the pose of the smart device relative to the visual axis is continuously measured, it can be compensated for (“computationally extracted”), such that distance measurements between camera sensor and the eye(s) with different tilt angles become comparable. A linkage of this aspect with the lighting situation is additionally possible since the smart device is tilted more often in order to avoid reflections on the display or “to move away” from the text to be read.

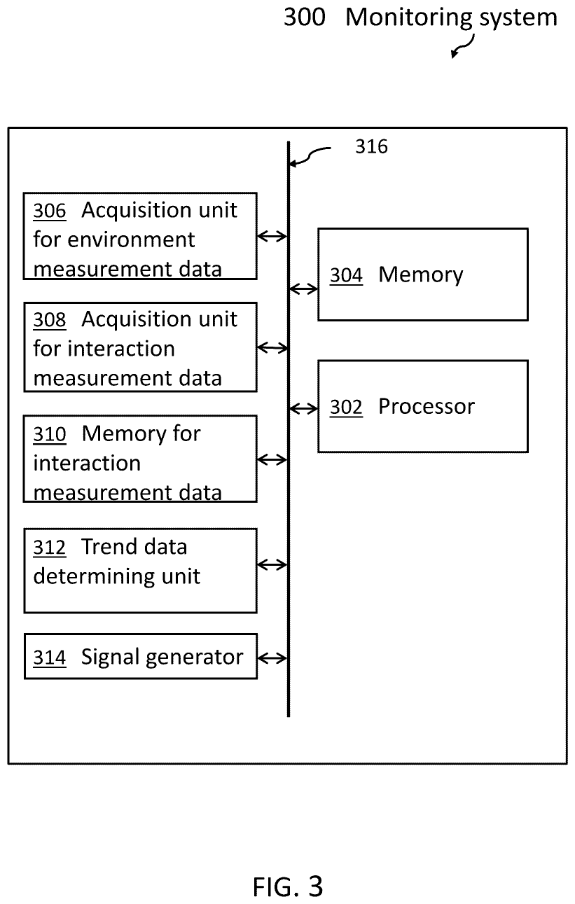

FIG. 3 shows a diagram of one exemplary embodiment of the monitoring system 300 for monitoring vital data of a user of a mobile device. The monitoring system 300 comprises a processor 302 and a memory 304 which operatively cooperates with the processor 302 to store instructions which, when executed by the processor 302, cause the processor 302 to implement: acquiring—in particular by way of a context acquisition unit 202 or acquisition unit for environment measurement data 306—environment measurement data in relation to the mobile device by means of a sensor that is fixedly integrated in the mobile device, and acquiring interaction measurement data—in particular by way of an acquisition unit for interaction measurement data 308 (cf. interaction acquisition unit 210, FIG. 2)—of the user with the mobile device by means of a sensor that is fixedly integrated in the mobile device (i.e. the smart device 204). In this case, the interaction measurement data should be acquired only if predefined conditions in relation to the acquired environment measurement data are met.

The monitoring system 300 additionally comprises a memory 212 which is useful for storing the interaction measurement data. Moreover, environment measurement data can additionally be stored here. Furthermore, the environment measurement data can also be stored in a dedicated memory.

The monitoring system 300 furthermore comprises a trend data determining unit, 312, in order to ascertain trend data in the interaction measurement data for a configurable time period on the basis of a trend analysis (cf. FIG. 2, 214).

Finally, the signal generator 314 of the monitoring system 300 serves to output the abovementioned signal, under previously defined conditions. The signal is at least output here if one of the following conditions is met: (i) exceedance or undershooting of a first configurable threshold value regarding the determined trend data or the directly measured interaction measurement data and/or (ii) exceedance or undershooting of a second configurable threshold value regarding one of the acquired interaction measurement data, or that of corresponding trend data was exceeded, in order to monitor the vital data of the user.

Express reference is made to the fact that the modules and units—in particular the processor 302, the memory 304, the acquisition unit for environment measurement data 306, the interaction measurement data 308, the memory 310 for the interaction measurement data, the trend data determining unit 312 and the signal generator 314—can be connected to electrical signal lines or via a system-internal bus system 316 for the purpose of exchanging signals or data.

Moreover, it is mentioned that the presented monitoring system is realizable from a combination of a smart device with a corresponding app. However, this does not preclude the use of dedicated electronic circuits for the abovementioned components and modules.

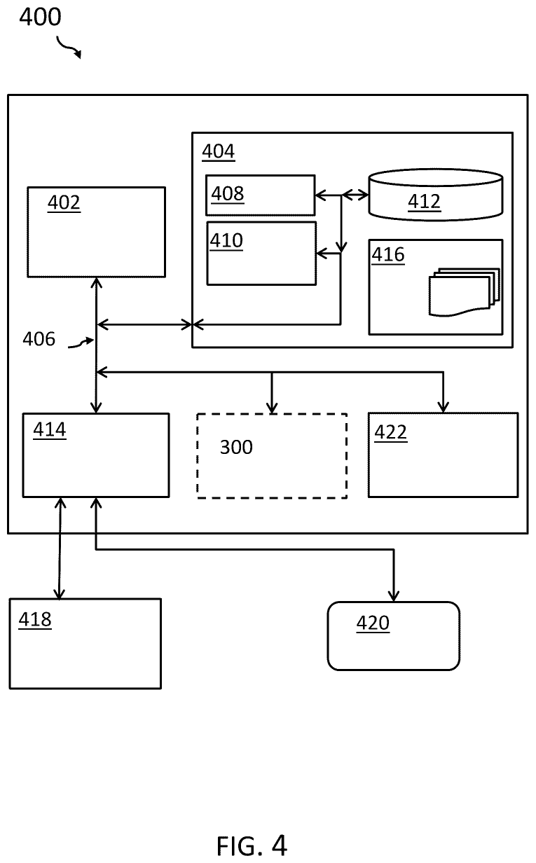

FIG. 4 illustrates one exemplary embodiment of a computer system 400 comprising the monitoring system 300 according to FIG. 3. The computer system 400 has a plurality of general-purpose functions. The computer system can in this case be a tablet computer, a laptop/notebook computer, another portable or mobile electronic device, a microprocessor system, a microprocessor-based system, a smartphone, a computer system with specifically configured special functions or else a constituent part of a microscope system. The computer system 400 can be configured to execute computer system-executable instructions—such as, for example, program modules—which can be executed in order to implement functions of the concepts proposed here. For this purpose, the program modules can comprise routines, programs, objects, components, logic, data structures, etc. in order to implement particular tasks or particular abstract data types.

The components of the computer system which can be jointly used at least in part by elements of the monitoring system 300 can comprise the following: one or more processors or processing units 402, a storage system 404 and a bus system 406 that connects various system components, including the storage system 404, to the processor 402. The computer system 400 typically comprises a plurality of volatile or nonvolatile storage media accessible by the computer system 400. The storage system 404 can store the data and/or instructions (commands) of the storage media in volatile form—such as for example in a RAM (random access memory) 408—in order to be executed by the processor 402. These data and instructions realize one or more functions and/or steps of the concept presented here. Further components of the storage system 404 can be a permanent memory (ROM) 410 and a long-term memory 412, in which the program modules and data (reference sign 416) and also workflows can be stored.

The computer system comprises a number of dedicated apparatuses (keyboard 418, mouse/pointing device (not illustrated), screen 420, etc.) for communication purposes. These dedicated devices can also be combined in a touch-sensitive display. An I/O controller 414, provided separately, ensures a frictionless exchange of data with external devices. A network adapter 422 is available for communication via a local or global network (LAN, WAN, for example via the Internet). The network adapter can be accessed by other components of the computer system 400 via the bus system 406. It is understood in this case—although it is not illustrated—that other devices can also be connected to the computer system 400.

In addition, at least parts of the monitoring system 300 for monitoring vital parameters of a user (cf. FIG. 3) can be connected to the bus system 406. The training data generator system 400 and the computer system 400 can optionally use the memories and/or the processors 402 jointly.

What is claimed is:

1. A computer-implemented method for monitoring vital data of a user of a mobile device, the method comprising
acquiring environment measurement data in relation to the mobile device using a sensor that is fixedly integrated in the mobile device,
acquiring interaction measurement data of the user with the mobile device using a sensor, wherein the sensor is fixedly integrated in the mobile device, and wherein the interaction measurement data are acquired if predefined conditions in relation to the acquired environment measurement data are met,
storing the interaction measurement data,
determining trend data on the basis of the acquired interaction measurement data for a configurable time period, and
generating a signal when a condition is met which is selected from
exceedance of a first configurable threshold value regarding the determined trend data,
exceedance of a second configurable threshold value regarding one of the acquired interaction measurement data,
in order to monitor the vital data of the user.

2. The method as claimed in claim 1, furthermore comprising
reducing the interaction measurement data on the basis of a rule.

3. The method as claimed in claim 1, wherein the acquired interaction measurement data are indicative of physical and/or mental vital parameter values of the user.

4. The method as claimed in claim 1, wherein the interaction measurement data comprise at least one from the group consisting of the following:
amplitude of an audio signal of the mobile device,
frequency distribution of an audio signal of the mobile device,
distance between the mobile device and the user's head during an active use of the mobile device,
brightness of a screen signal of the mobile device,
frequency of eye blinking by the user,
speed of the user's reaction to optical stimuli originating from the mobile device,
attention duration,
text input speed,
number of actively used apps,
rectilinearity of acquired swiping movements,
frequency of use of the mobile device,
screen areas of the mobile device used by the user, and
zoom settings.

5. The method as claimed in claim 1, wherein the environment measurement data comprise at least one from the group consisting of the following:
ambient brightness values,
ambient volume values,
kind of represented information of the mobile device,
spatial orientation of the mobile device,
speed of movement of the mobile device,
movement profile of the mobile device,
GPS coordinate values, and
camera image.

6. The method as claimed in claim 1, additionally comprising
allocating a predefined profile of interaction measurement data to at least one characteristic of vital parameter values.

7. The method as claimed in claim 1, moreover comprising
actively instructing the user, by means of the mobile device, to fulfil a predefined use profile in relation to the mobile device if predefined interaction measurement data have not been acquired within a predefined time interval.

8. The method as claimed in claim 7, wherein actively instructing takes place if the environment measurement data for the acquisition of the interaction measurement data are in a predefined value range.

9. The method as claimed in claim 1, additionally comprising
reducing a number of interaction measurement data assigned to one another if
a change in interaction measurement data assigned to one another is below a predefined threshold value, or
after a representative value has been determined for a stored amount of interaction measurement data that have been ascertained within a period of time, or
after a configurable time period has elapsed.

10. The method as claimed in claim 1, furthermore comprising
acquiring an input of a situation indication into the mobile device, wherein the situation indication is selected from a selection of situation indications provided by the mobile device.

11. The method as claimed in claim 1, wherein acquiring the interaction measurement data also comprises
quasi-continuously measuring a distance between the screen of the mobile device and an eye of the user within a predefined time period and/or while previously determined environment measurement data values are present, and
using different tilt angles of the mobile device for determining a reference distance between at least one eye of the user and the screen surface.

12. The method as claimed in claim 1, furthermore comprising
determining a quality value of the acquired interaction measurement data, and
repeating the acquisition of the interaction measurement data if the quality value is below a predefined quality reference value.

13. The method as claimed in claim 1, furthermore comprising
using an acquisition profile for acquiring the environment measurement data and/or acquiring the interaction measurement data.

14. A monitoring system for monitoring vital data of a user of a mobile device, the monitoring system comprising
a processor,
a memory which operatively cooperates with the processor to store instructions which, when executed by the processor, cause the processor to implement
acquiring environment measurement data in relation to the mobile device by means of a sensor that is fixedly integrated in the mobile device,
acquiring interaction measurement data of the user with the mobile device by means of a sensor, wherein the sensor is fixedly integrated in the mobile device, and wherein the interaction measurement data are acquired if predefined conditions in relation to the acquired environment measurement data are met,
storing the interaction measurement data,
determining trend data on the basis of the acquired interaction measurement data for a configurable time period, and
generating a signal when a condition is met which is selected from
exceedance of a first configurable threshold value regarding the determined trend data, and
exceedance of a second configurable threshold value regarding one of the acquired interaction measurement data,
in order to monitor the vital data of the user.

15. A computer program product for monitoring vital data of a user of a mobile device, wherein the computer program product comprises a computer-readable storage medium comprising program instructions stored thereon, wherein the program instructions are executable by one or more computers or control units and cause said one or more computers or control units to carry out the method as claimed in claim 1.
